(19) United States
(12) Patent Application Publication
(10) Pub. No.: US 2015/0316546 A1
(43) Pub. Date: Nov. 5, 2015

(54) SENSOR CHIP

(71) Applicant: PANASONIC INTELLECTUAL PROPERTY MANAGEMENT CO., LTD., Osaka-shi,Osaka (JP)

(72) Inventors: TAKUYA OKA, Kyoto (JP); MAKOTO TAKAHASHI, Hyogo (JP); ATSUSHI SHUNORI, Osaka (JP)

(21) Appl. No.: 14/652,424

(22) PCT Filed: Dec. 4, 2013
(86) PCT No.: PCT/JP2013/007108

§ 371 Date: Jun. 15, 2015

(30) Foreign Application Priority Data
Dec. 20, 2012 (JP) 2012-277783

(51) Int. Cl. G01N 33/543

(52) U.S. Cl. CPC G01N 33/54386

(57) ABSTRACT

A sensor chip is configured to be used together with probe solution containing a probe for capturing an object substance. The sensor chip includes a substrate, a probe-immobilizing part disposed on an upper surface of the substrate, and a liquid-spread-prevention part disposed around the probe-immobilizing part. The probe-immobilizing part is configured to immobilize the probe by allowing the probe solution to be spotted to the probe-immobilizing part. The liquid-spread-prevention part prevents a liquid from spreading from the probe-immobilizing part. The probe-immobilizing part is made of a porous material having pores provided therein. This sensor chip has high detection sensitivity.

### TECHNICAL FIELD

The present invention relates to a sensor chip used for detecting or analyzing biological samples, such as nucleic acids, protein material, sugar chain, or lipid.

### BACKGROUND ART

A microarray chip, an application of a conventional sensor chip, is a device capable of simultaneously detecting multiple substances, for example, nucleic acid molecules and protein. As an example of the microarray chip, a DNA microarray detects DNA molecules. DNA microarrays are formed such that a nucleic acid as a probe is immobilized onto a flat surface of a glass slide or a silicon substrate. Hybridization reaction of the probe with a sample containing an object substance, such as nucleic acid molecules, a DNA or RNA specific to the base sequence of the nucleic acid as a probe can be detected. Many kinds of DNAs and RNAs are detected simultaneously by immobilizing such a probe onto a flat surface of glass slide in an array. This provides DNA analysis with high efficiency. DNA microarrays are used extensively not only in the area of research but also in the area of clinic diagnosis.

Protein arrays made of resin having cellulose nitrate, nylon, and polyvinylidene difluoride are also employed for the flat surface on which a probe is immobilized. A protein array employs an antibody and recombinant protein as a probe. Through reaction of the probe with an object substance, such as a viral antigen, a hormone of protein, and a peptide, many kinds of substances are simultaneously detected. As for the protein arrays, the hydrophobic property of the resin surface allows a probe to be easily and efficiently immobilized thereon, and therefore it is favorably used in proteomics research (for example, see PTL 1).

The quantity and density of the immobilized probe have an influence on sensitivity for detecting an object substance. Therefore, an analysis that requires high detection accuracy needs rigorously-controlled quantity and density of the probe. For example, Patent Literature 2 discloses a method for immobilizing probes in an array. According to the method, probe solution in which a probe is mixed with a probe-immobilizing substance is spotted onto the surface of the sensor chip (see PTL 2).

For example, PTL 3 discloses a sensor chip in which various kinds of probes are immobilized in an array by applying a probe onto an activated and hydrophobized surface of the sensor chip.

However, in a method of producing a sensor chip where probe immobilization is obtained by spotting, the spotting diameter can be affected by a surface tension of a sample to be used. That is, the surface tension of solution varies with difference in probe density or in composition of probe solutions, which changes the contact area between the solution and the surface. As a result, the spotting diameters have lack of uniformity when various kinds of probes are immobilized in an array as is in a microarray. That is, uniformity in spotting diameters is required in probe immobilization employing probe solutions of different kind or different density. Besides, the spot size is affected by hygrothermal condition in spotting operation. Therefore, a method capable of providing reproducibly-obtained uniform diameter in spotting has been required.

### CITATION LIST

### Patent Literature

PTL 1: Japanese Patent Laid-Open Publication No. 2005-504309

PTL 2: Japanese Patent No. 4532854

PTL 3: Japanese Patent No. 4209494

### SUMMARY

A sensor chip is configured to be used together with probe solution containing a probe for capturing an object substance. The sensor chip includes a substrate, a probe-immobilizing part disposed on an upper surface of the substrate, and a liquid-spread-prevention part disposed around the probe-immobilizing part. The probe-immobilizing part is configured to immobilize the probe by allowing the probe solution to be spotted to the probe-immobilizing part. The liquid-spread-prevention part prevents a liquid from spreading from the probe-immobilizing part. The probe-immobilizing part is made of a porous material having pores provided therein.

This sensor chip has high detection sensitivity.

### BRIEF DESCRIPTION OF DRAWINGS

FIG. 1 is a cross-sectional view of a sensor chip in accordance with Exemplary Embodiment 1 of the present invention.

FIG. 2 is a cross-sectional view the sensor chip in accordance with Embodiment 1 for illustrating a method of immobilizing a probe onto the sensor chip.

FIG. 3 is a cross-sectional view of the sensor chip having a probe immobilized thereon in accordance with Embodiment 1.

FIG. 4 is a cross-sectional view of the sensor chip in accordance with Embodiment 1 for illustrating another method of immobilizing a probe onto the sensor chip.

FIG. 5 is a cross-sectional view of the sensor chip with a probe immobilized thereon in accordance with Embodiment 1.

FIG. 6A is a cross-sectional view of the sensor chip in accordance with Embodiment 1 for illustrating a method of producing the sensor chip and a method of immobilizing a probe.

FIG. 6B is a cross-sectional view of the sensor chip in accordance with Embodiment 1 for illustrating the method of producing the sensor chip and the method of immobilizing a probe.

FIG. 6C is a cross-sectional view of the sensor chip in accordance with Embodiment 1 for illustrating the method of producing the sensor chip and the method of immobilizing a probe.

FIG. 6D is a cross-sectional view of the sensor chip in accordance with Embodiment 1 for illustrating the method of producing the sensor chip and the method of immobilizing a probe.

FIG. 6E is a cross-sectional view of the sensor chip in accordance with Embodiment 1 for illustrating the method of producing the sensor chip and the method of immobilizing a probe.

FIG. 7 is a cross-sectional view of another sensor chip in accordance with Embodiment 1.

FIG. 8A is a cross-sectional view of a sensor chip in accordance with Exemplary Embodiment 2 of the present invention.

FIG. 8B is a cross-sectional view of another sensor chip in accordance with Embodiment 2.

FIG. 8C is a cross-sectional view of still another sensor chip in accordance with Embodiment 2.

FIG. 9 is a cross-sectional view of a sensor chip in accordance with Exemplary Embodiment 3 of the present invention.

FIG. 10 is a cross-sectional view of the sensor chip in accordance with Embodiment 3 for illustrating a method of immobilizing a probe onto the sensor chip.

FIG. 11 is a perspective top view of another sensor chip in accordance with Embodiment 3.

FIG. 12 is an enlarged view of the sensor chip in accordance with Embodiment 3 for showing sample reaction observed in the sensor chip.

FIG. 13 is a cross-sectional view of the sensor chip in accordance with Embodiment 3 for showing a detection method with use of the sensor chip.

FIG. 14A is a cross-sectional view of the sensor chip in accordance with Embodiment 3 for illustrating a method of producing the sensor chip.

FIG. 14B is a cross-sectional view of the sensor chip in accordance with Embodiment 3 for illustrating a method of producing the sensor chip.

FIG. 14C is a cross-sectional view of the sensor chip in accordance with Embodiment 3 for illustrating a method of producing the sensor chip.

FIG. 15 is a cross-sectional view of another sensor chip in accordance with Embodiment 3.

### DETAIL DESCRIPTION OF PREFERRED EMBODIMENTS

### Exemplary Embodiment 1

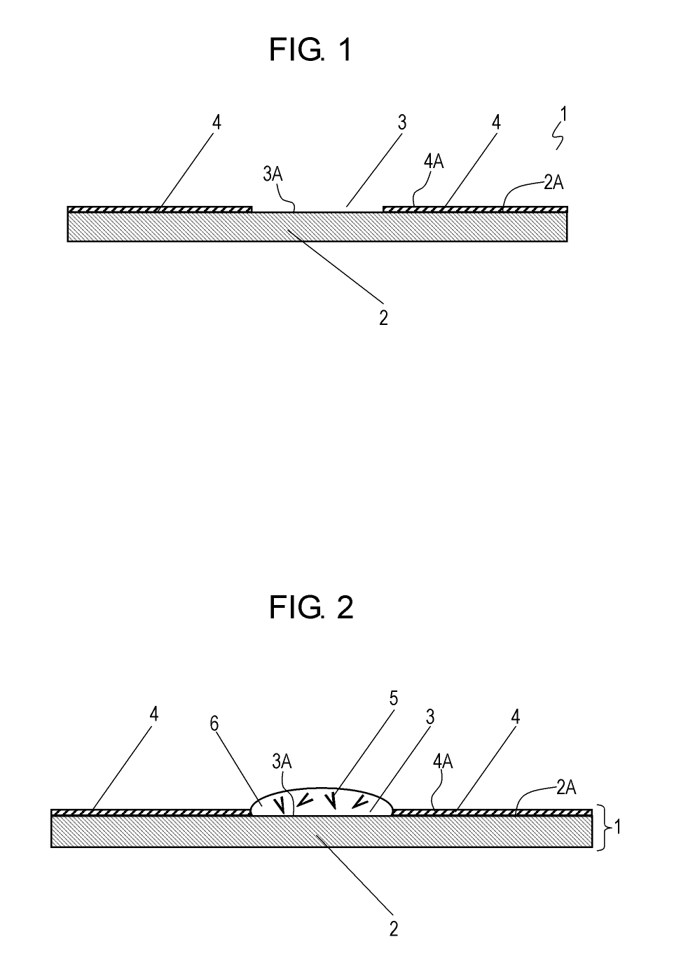

FIG. 1 is a cross-sectional view of a sensor chip in accordance with Exemplary Embodiment 1 of the present invention. Sensor chip 1 includes substrate 2, probe-immobilizing part 3 disposed on upper surface 2A of substrate 2, and liquid-spread-prevention part 4 disposed on upper surface 2A of substrate 2.

Probe-immobilizing part 3 is configured to immobilize a probe thereon for capturing an object substance. The object substance is a substance that specifically reacts with the probe, i.e., biological samples, such as nucleic acids, protein, sugar chain, or lipid.

Liquid-spread-prevention part 4 is disposed around probe-immobilizing part 3 so as to prevent liquid spotted to probe-immobilizing part 3 from spreading from probe-immobilizing part 3.

Substrate 2 may be made of inorganic material, such as glass, silicon, quartz, ceramics, silicon dioxide, or metal, resin, such as COC, COP, PC, PMMA, SAN, or PS, or organic material. In terms of flatness of the surface including upper surface 2A, a quartz plate having a surface which has unevenness not greater than 5 μm is preferably employed for substrate 2.

Probe-immobilizing part 3 may be upper surface 2A itself of substrate 2. Further, probe-immobilizing part 3 may be a chemical functional group applied onto upper surface 2A of substrate 2.

For example, to apply a silanol group onto upper surface 2A of substrate 2, upper surface 2A undergoes surface modification by a silane-coupling agent so that a silanol group can be applied to upper surface 2A.

To apply an epoxide group onto upper surface 2A of substrate 2, for example, the following procedures are carried out. First, 3-Glycidoxypropyltrimethoxysilane is stirred into 2% acetic acid aqueous solution so as to keep hydrolysis reaction for a period of time ranging from 30 minutes to one hour. After that, the solution is dripped onto upper surface 2A of substrate 2 and is kept reaction for a period of time not less than 30 minutes at room temperature. Through the procedures above, an epoxide group is applied onto upper surface 2A of substrate 2. When a protein, such as an antibody, is employed for the probe, the epoxide group on upper surface 2A binds covalently to the probe by dehydration condensation, so that the probe can be immobilized onto probe-immobilizing part 3.

With use of a silane-coupling agent, for example, the following chemical functional groups can be applied to upper surface 2A of substrate 2: an isocyanate group; a carboxyl group; a methacryloxy group; an amino group; an acrylic group; a vinyl group; an aldehyde group; and a maleimide group. According to the type of the probe, an optimal surface treatment is provided to the substrate.

Liquid-spread-prevention part 4 is, for example, disposed in parallel to upper surface 2A of substrate 2. Liquid-spread-prevention part 4 disposed in parallel to upper surface 2A allows probe-immobilizing part 3 to be parallel to upper surface 2A of substrate 2. This configuration allows plural probes to simultaneously detect easily by scanning the same plane.

Liquid-spread-prevention part 4 may undergo surface treatment to have hydrophobicity with use of a surface preparation agent, such as water-repellent coating, having high hydrophobicity. For example, with use of n-octadecyltrichlorosilane as a surface preparation agent with high hydrophobicity, straight-chain carbon hydride applied to upper surface 2A of substrate 2 provides liquid-spread-prevention part 4 with water repellency. However, a surface preparation agent for applying hydrophobicity is not limited to the aforementioned agent; for example, a hydrophobic surface preparation agent such as a fluorine compound may be similarly employed. For increasing hydrophobicity, a surface preparation agent capable of forming an angle of contact (between water and the surface) exceeding 80° may preferably be used; further, forming an angle of contact exceeding 150° may be more preferable. On such an extremely water-repellent surface with an angle of contact exceeding 150°, a noise caused by nonspecific adsorption in the reaction of probe 5 with an object substance is suppressed, accordingly increasing detection sensitivity of sensor chip 1.

Liquid-spread-prevention part 4 may undergo one or more surface treatments. Such structured liquid-spread-prevention part 4 maintains its flatness, eliminating influence due to unevenness on detection sensitivity.

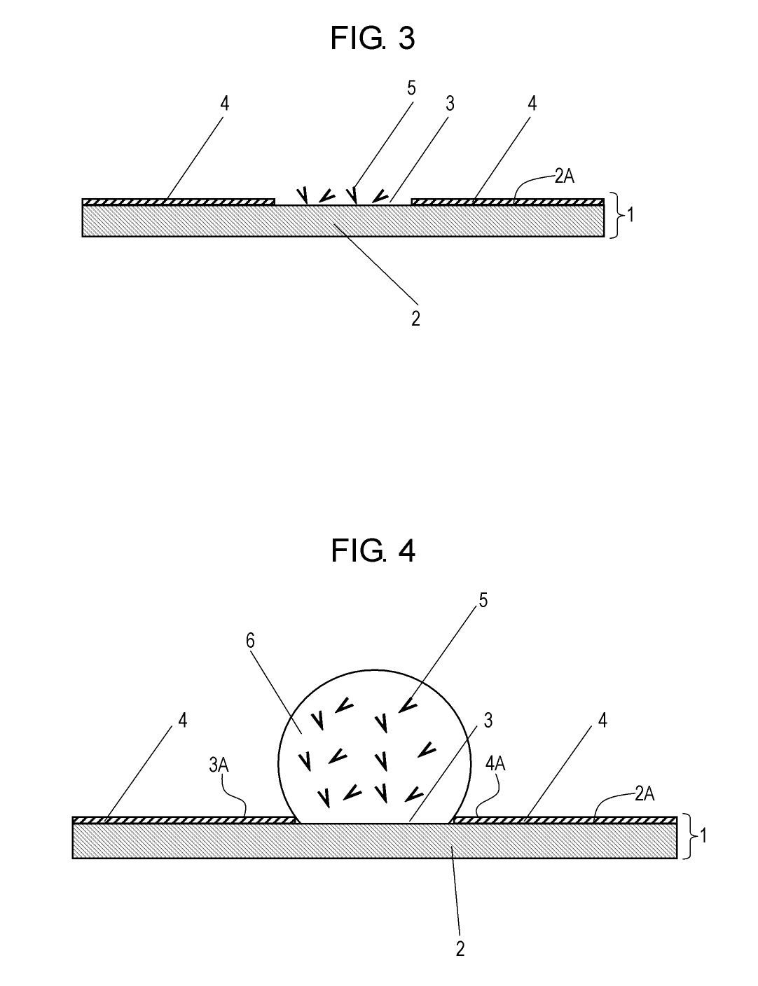

FIG. 2 is a cross-sectional view of sensor chip 1 in accordance with Embodiment 1 for illustrating a method of immobilizing probe 5 onto sensor chip 1. FIG. 3 is a cross-sectional view of sensor chip 1 having probe 5 immobilized thereon.

As shown in FIG. 2, probe solution 6 containing probe 5 is dripped onto upper surface 3A of probe-immobilizing part 3. As probe 5, for example, a protein, such as an antibody or a receptor, that has selective bonding with a specific substance, nucleic acids or a substance similar to nucleic acids, in which the parts having complementary sequence are hybridized, are employed. However, probe 5 is not limited to the biological polymers described above; probe 5 may be a molecule that has specific bonding with an intended object substance. For example, a low-molecular compound obtained by combinatorial chemistry for probe 5 allows the sensor chip to search ligands.

As probe solution 6, for example, the following is used: a buffer solution that contains polyoxethylene sorbitan monolaurate as a nonionic surface-active agent and has probe 5 suspended therein. Specifically, for example, a phosphate buffer solution that contains a surface-active agent and 1 mg/mL to 1 mg/mL of a monoclonal antibody suspended therein.

Probe solution 6 is not limited to the surface-active agents above. To stabilize probe 5, polysaccharide, such as trehalose, and an antiseptic agent, such as NaN₃, may be used. Further, to enhance the bonding between probe 5 and probe-immobilizing part 3, the following active substances may be employed: N-hydroxysuccinimideester (NHS), 1-Ethyl-3-(3-dimethylaminopropyl) carbodiimide (WSC).

For example, probe solution 6 may be applied by a constant amount onto upper surface 2A of substrate 2 with use of a micro dispenser of an ink-jet mechanism. Probe-immobilizing part 3 may preferably have a circular shape with a diameter ranging from 10 to 1000 μm; more preferably, ranging from 10 to 200 μm. The amount of probe solution 6 to be discharged onto probe-immobilizing part 3 ranges from 1 pL to 100 nL.

Probe-immobilizing part 3 formed as a spot with a diameter not smaller than 10 μm allows an ordinarily-used laser scanner to perform detection accurately. Further, being formed as spots each having a diameter not larger than 200 μm or smaller, probe-immobilizing parts 3 of two hundred to three hundred spots can be arranged in the same reaction section disposed at intervals of nine millimeters generally used Society for Biomolecular Screening (SBS) format. This configuration allows the probes immobilized to each probe-immobilizing part 3 to easily perform simultaneous multi-item detection.

In the structure above, liquid-spread-prevention part 4 repels probe solution 6, and therefore, the surface which probe solution 6 contacts is limited to probe-immobilizing part 3. As a result, as shown in FIG. 3, probe 5 is immobilized not onto upper surface 4A of liquid-spread-prevention part 4, but only onto probe-immobilizing part 3.

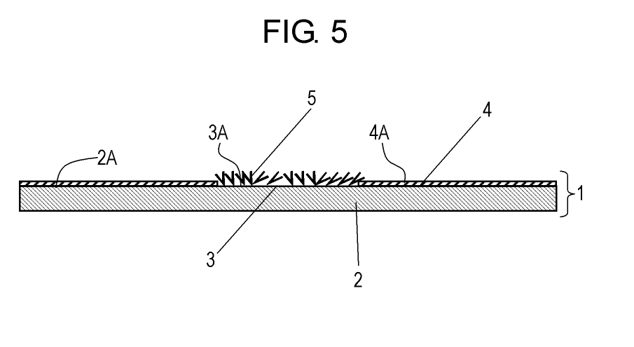

FIG. 4 is a cross-sectional view of sensor chip 1 in accordance with Embodiment 1 for illustrating the method of immobilizing the probe in a case that probe solution of a large amount is applied to the sensor chip. FIG. 5 is a cross-sectional view of sensor chip 1 having the probe immobilized thereon in accordance with Embodiment 1 in the case that probe solution of a large amount is applied to the sensor chip. The spotting amount of probe solution 6 may be increased to increase the amount of probe 5 to be immobilized onto probe-immobilizing part 3. In this case, liquid-spread-prevention part 4 blocks probe solution 6 so as to prevent the solution from spreading outside probe-immobilizing part 3. Therefore, as shown in FIG. 5, probe 5 is immobilized not onto upper surface 4A of liquid-spread-prevention part 4, but only onto probe-immobilizing part 3.

The discharge amount of probe solution 6 is affected by viscosity of probe solution 6. To maintain a reproducibly-controlled discharge amount, probe 5 has a constant concentration in probe solution 6. In a case that the sensor chip has no liquid-spread-prevention part 4, when the discharge amount of probe solution 6 is increased so as to increase probe 5 immobilized onto the surface, the contact area of the spot on substrate 2 increases accordingly, increasing the area in which probe 5 is immobilized accordingly.

When probe solution 6 is a liquid easily spreading, i.e., a liquid with a small surface tension, it is difficult to decrease the spot diameter. The method in which the spot diameter is determined in accordance with Embodiment 1 is effectively used for analysis of a diagnosis that requires uniformity.

Lack of uniformity in diameters of the spots causes lack of uniformity in density of immobilized probe 5. This also causes lack of uniformity in signal intensity obtained by reaction of probe 5 with an object substance, resulting in poor reproducibility of the test. As shown in FIG. 5, immobilizing probe 5 onto only predetermined probe-immobilizing part 3 enhances reproducibility of intended signal intensity.

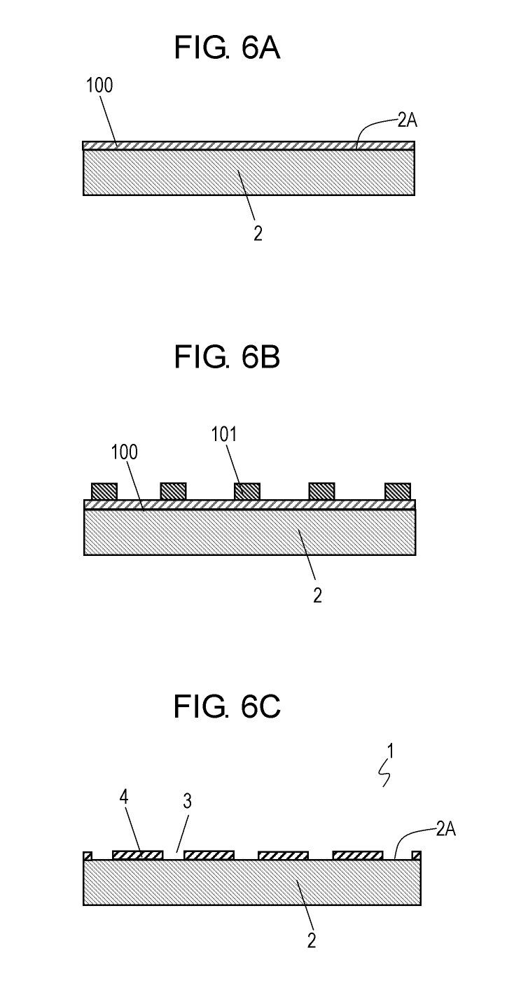

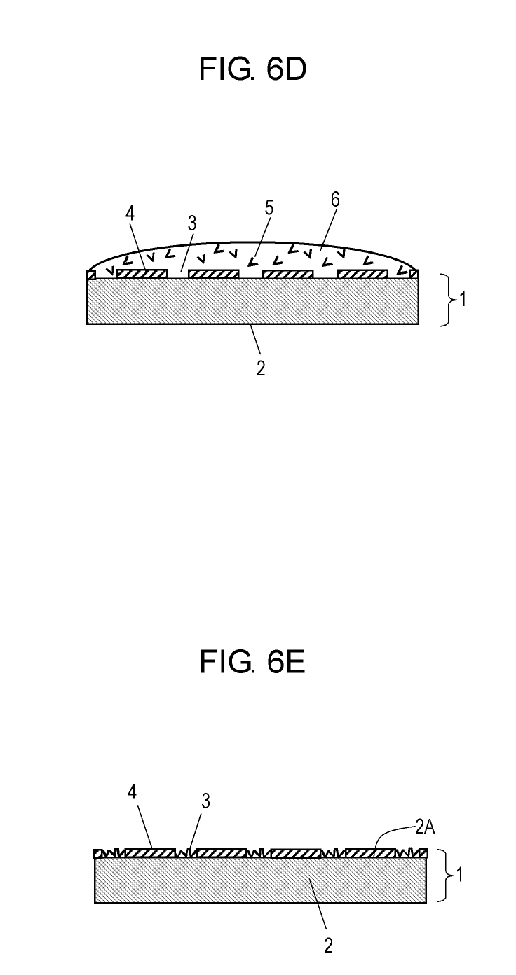

Liquid-spread-prevention part 4 can be formed by a photolithographic method. FIGS. 6A to 6E are cross-sectional views of sensor chip 1 according to Embodiment for illustrating a method of producing sensor chip 1, specifically illustrating a procedure for immobilizing probe 5.

First, substrate 2 is prepared. A glass slide is employed for substrate 2 in accordance with the embodiment. Upper surface 2A of substrate 2 is coated with an aqueous solution of photocrosslinking polymer 100. To obtain the aqueous solution of photocrosslinking polymer 100, for example, BIOSURFINE-AWP (made by Toyo Gosei Co., Ltd) is diluted with pure water so as to obtain 1.2% aqueous solution. The solution is applied over upper surface 2A of substrate 2 with a spin coater by spin-coating with 1000 rpm for 30 seconds. This spin-coating forms photocrosslinking polymer 100 with a film thickness of approximately 0.8 μm on upper surface 2A of substrate 2, as shown in FIG. 6A.

Next, as shown in FIG. 6B, mask 101 is placed on the upper surface of photocrosslinking polymer 100, and the surface having mask 101 thereon is irradiated with ultraviolet to have photocrosslinking. To be specific, masks 101 with a diameter of 200 μm arranged by a pitch distance of 400 μm are placed on the surface of photocrosslinking polymer 100, and 100-mJ ultraviolet light is applied over mask 101 so that photocrosslinking polymer 100 may have photocrosslinking.

After the UV irradiation, substrate 2 is immersed in pure water for one minute. After that, uncured polymer 100 on the surface is removed by washing, and then, substrate 2 is dried with dry air. Through the UV curing above, as shown in FIG. 6C, liquid-spread-prevention part 4 is formed. Liquid-spread-prevention part 4 is made of a cured resin coat with water resistance. The part between liquid-spread-prevention parts 4, which polymer 100 is removed from by washing, functions as probe-immobilizing part 3. Next, as shown in FIG. 6D, probe solution 6 is dripped on upper surface 2A having probe-immobilizing part 3 of substrate 2 so as to immobilize probe 5. For example, streptavidinCy3 with a dilution ratio of 2.5 μg/ml is dripped onto the surface, and then substrate 2 is let stand for five minutes at room temperature. After that, substrate 2 is immersed in pure water for one minute, then rinsed, and dried by dry air. Through the procedures above, probe 5 is immobilized to probe-immobilizing part 3, as shown in FIG. 6E.

Probe-immobilizing parts 3 have a uniform diameter of 200 μm. Further, a mean fluorescence intensity of sensor chip 1 is calculated with a laser scanner. The mean fluorescence intensity at liquid-spread-prevention part 4 measured 309, and the mean fluorescence intensity at probe-immobilizing part 3 measured 1113.

As described above, liquid-spread-prevention part 4, formed by photolithography, for blocking the spreading of probe solution 6 allows probe-immobilizing parts 3 to have a uniform diameter.

As described above, sensor chip 1 is configured to be used together with probe solution 6 containing probe 5 for capturing an object substance. Sensor chip 1 includes substrate 2, probe-immobilizing part 3 disposed on upper surface 2A of substrate 2, and liquid-spread-prevention part 4 disposed around probe-immobilizing part 3 and on upper surface 2A of substrate 2. Probe-immobilizing part 3 is configured to immobilize probe 5 by allowing probe solution 6 to be spotted to probe-immobilizing part 3. Liquid-spread-prevention part 4 prevents the spotted solution from spreading from probe-immobilizing part 3.

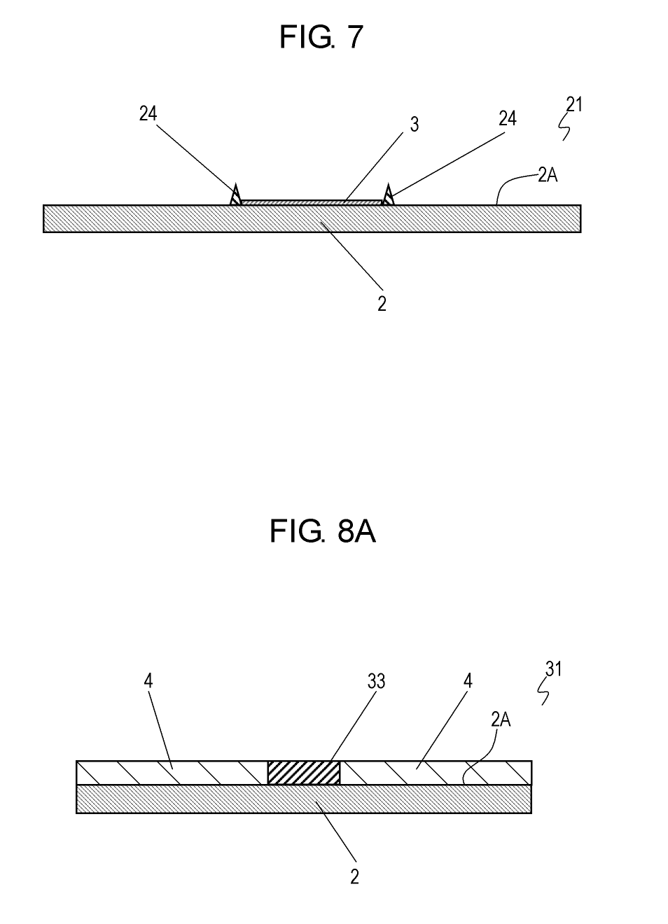

FIG. 7 is a cross-sectional view of another sensor chip 21 according to Embodiment 1. In FIG. 7, components identical to those of sensor chip 1 shown in FIG. 1 are denoted by the same reference numerals. Sensor chip 21 shown in FIG. 7 includes liquid-spread-prevention part 24 on upper surface 2A of substrate 2 instead of liquid-spread-prevention part 4 of sensor chip 1. Liquid-spread-prevention part 24 protrudes from upper surface 2A of substrate 2.

Liquid-spread-prevention part 24 is made of the same material as substrate 2. For example, in the case that substrate 2 is made of resin material, substrate 2 having liquid-spread-prevention part 24 is formed by metallic molding, injection molding, or cutting molding. The height of liquid-spread-prevention part 24 protruding from upper surface 2A of substrate 2 ranges from 1 to 100 μm to provide probe-immobilizing part 3 with predetermined outer shape and area.

In the case that protruding liquid-spread-prevention part 24 is made of inorganic material, liquid-spread-prevention part is produced in a process for where chemical etching or cutting molding substrate 2.

The height of protruding liquid-spread-prevention part 24 may ranges preferably from 10 μm to 1 mm. The height of Protruding liquid-spread-prevention part 24 not less than 10 μm allows probe-immobilizing part 3 with a circular shape with a diameter of 200 μm to capture a droplet of sub-nL discharged with a generally used micro droplet discharge device. Protruded liquid-spread-prevention part 24 with a height not larger than 1 mm allows a generally used fluorescence scanner to smoothly scan probe-immobilizing part 3 and liquid-spread-prevention part 24 with no hindrance.

Liquid-spread-prevention part 24 may have a fibrous structure directly bonded to upper surface 2A of substrate 2. For example, a fibrous structure formed directly on upper surface 2A of substrate 2 by chemical deposition or vapor-phase deposition. Further, surface treatment may be provided on the surface of the fibrous structure with use of a surface preparation agent to apply hydrophobicity.

With the structure above, probe solution 6 is captured only by probe-immobilizing part 3. Even when the amount of probe solution 6 changes, the structure provides the uniform areas having probe 5 immobilized thereon, and uniformizes signal intensity obtained by antigen-antibody reaction of sensor chip 21.

### Exemplary Embodiment 2

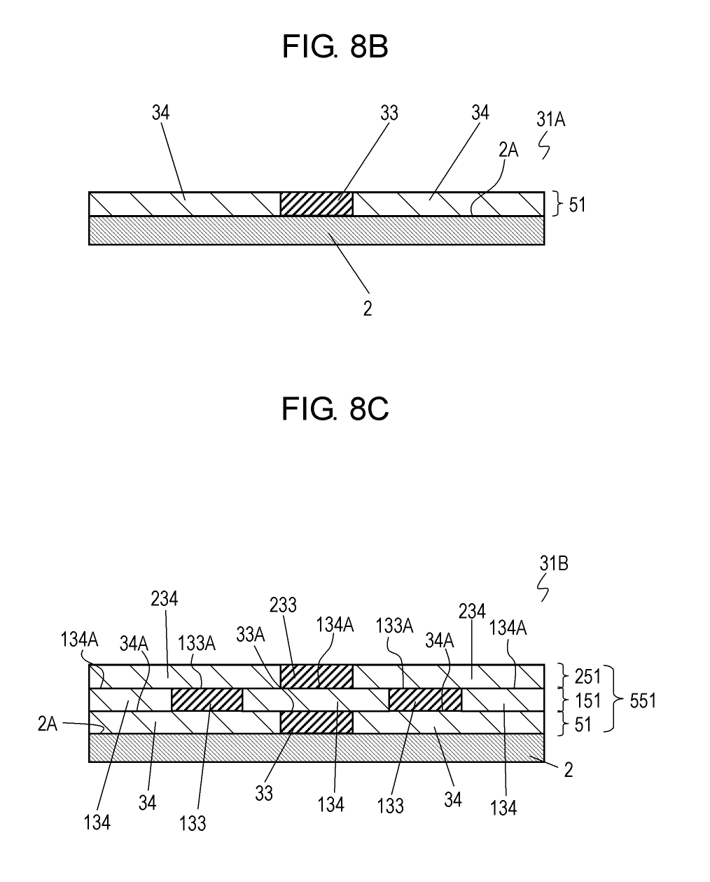

FIG. 8A is a cross-sectional view of sensor chip 31 in accordance with Exemplary Embodiment 2 of the present invention. In FIG. 8A, components identical to those of sensor chip 1 shown in FIG. 1 in accordance with Embodiment 1 are denoted by the same reference numerals. Sensor chip 31 according to Embodiment 2 includes has probe-immobilizing part 33 made of porous body instead of probe-immobilizing part 3 of sensor chip 1 shown in FIG. 1 according to Embodiment 1.

As the porous body mentioned above, the following materials may be employed: a baked material of activated carbon, diatom earth, or porous silica with a film thickness ranging from 1 nm to 100 μm; a porous membrane material, such as cellulose nitrate; and a material of a base material, such as silicon, modified into porous formation by chemical etching.

Liquid-spread-prevention part 4 may not necessarily have a specific shape and a height, and may be disposed at a predetermined depth from upper surface 2A of substrate 2, or, for example, may be a protruding structure like liquid-spread-prevention part 24 of sensor chip 21 shown in FIG. 7 according to Embodiment 1. Besides, liquid-spread-prevention part 4 may be made of a porous body. As the porous body mentioned above, the following materials may be employed: a baked material of activated carbon, diatom earth, or porous silica with a film thickness ranging from 1 nm to 100 μm; a porous membrane material, such as cellulose nitrate; and a material of a base material, such as silicon, modified into porous formation by chemical etching.

The surface of liquid-spread-prevention part 4 may preferably have surface treatment provided thereon. For example, liquid-spread-prevention part 4 provided with water-repellent treatment allows probe 5 to be captured only on probe-immobilizing part 33. This configuration uniformizes the areas to which probe 5 is immobilized easily, even I the case that probe-immobilizing part 33 is made of a porous body.

In the case that liquid-spread-prevention part 4 is made of a porous body having pores therein, the water-repellent treatment may preferably be provided not only to the surface but also onto the inside of the body. Such treated prevention part 4 further prevents probe solution 6 from spreading.

As described above, sensor chip 31 is configured to be used together with probe solution 6 containing probe 5 for capturing an object substance. Sensor chip 31 includes substrate 2, probe-immobilizing part 33 disposed on upper surface 2A of substrate 2, and liquid-spread-prevention part 4 disposed around probe-immobilizing part 33 and on upper surface 2A of substrate 2. Probe-immobilizing part 33 is configured to immobilize probe 5 by allowing probe solution 6 to be spotted to probe-immobilizing part 33. Liquid-spread-prevention part 4 is configured to prevent the spotted solution from spreading from probe-immobilizing part 33. Probe-immobilizing part 33 is made of a porous body having pores therein.

FIG. 8B is a cross-sectional view of another sensor chip 31A according to Embodiment 2. In FIG. 8B, components identical to those of sensor chip 31 shown in FIG. 8A are denoted by the same reference numerals. Sensor chip 31A includes porous body 51 disposed on upper surface 2A of substrate 2. Probe-immobilizing part 33 and liquid-spread-prevention part 34 are implemented by single porous body 51. Liquid-spread-prevention part 34 has hydrophobicity, whereas probe-immobilizing part 33 has hydrophilicity. That is, liquid-spread-prevention part 34 has hydrophobicity higher than that of probe-immobilizing part 33. Porous body 51 having hydrophobic property constitutes liquid-spread-prevention part 34. On the other hand, porous body 51 having hydrophilic property constitutes probe-immobilizing part 33.

FIG. 8C is a cross-sectional view of still another sensor chip 31B according to Embodiment 2.In FIG. 8C, components identical to those of sensor chip 31A shown in FIG. 8B are denoted by the same reference numerals. Sensor chip 31B further includes porous body 151 disposed on upper surface 51A of porous body 51 of sensor chip 31A, and porous body 251 disposed on upper surface 151A of porous body 151. Porous body 151 includes probe-immobilizing part 133 having a property similar to probe-immobilizing part 33 of porous body 51 and liquid-spread-prevention part 134 having a property similar to liquid-spread-prevention part 34 of porous body 51. Similarly, porous body 251 includes probe-immobilizing part 233 having a property similar to probe-immobilizing part 33 of porous body 51 and liquid-spread-prevention part 234 having a property similar to liquid-spread-prevention part 34 of porous body 51. That is, probe-immobilizing parts 33, 133, and 233 are configured to immobilize probes by allowing the probe solution to be spotted to the probe-immobilizing parts. Liquid-spread-prevention parts 34, 134, and 234 are disposed around probe-immobilizing parts 33, 133, and 233, respectively, and are configured to prevent the spotted solution from spreading from probe-immobilizing part 33, 133, and 233, respectively. Liquid-spread-prevention parts 34, 134, and 234 have hydrophobicity, whereas probe-immobilizing parts 33, 133, and 233 have hydrophilicity. That is, liquid-spread-prevention parts 34, 134, and 234 have a hydrophobicity higher than that of probe-immobilizing parts 33, 133, and 233. Probe-immobilizing part 133 is located on upper surface 34A of liquid-spread-prevention part 34. Liquid-spread-prevention part 134 is located on upper surface 33A of probe-immobilizing part 33. Probe-immobilizing part 233 is located on upper surface 134A of liquid-spread-prevention part 134. Liquid-spread-prevention part 234 is located on upper surface 133A of probe-immobilizing part 133. Porous bodies 51, 151, and 251 are stacked to form porous body 551 as a single structure. The material of porous bodies 51, 151, and 251 may be identical to each other, or may be different from each other. Porous body 551 may not be an actually layered structure; it may be virtually regarded as a layered structure of porous bodies 51, 151, and 251. As described above, hydrophilic probe-immobilizing parts 33, 133, and 233 and hydrophobic liquid-spread-prevention parts 34, 134, and 234 are selectively arranged in a thickness direction of porous body 551 that has pores therein and is disposed on upper surface 2A of substrate 2. This configuration allows probe-immobilizing parts 33, 133, and 233 to be spatially disposed in the structure.

The spatial arrangement of probe-immobilizing parts 33, 133, and 233 forms the area for capturing an object substance in the thickness direction of sensor chip 31B, i.e., in a direction perpendicular to upper surface 2A of substrate 2, hence increasing the detection area without extending sensor chip 31B horizontally. This configuration increases the detection area per single chip, allowing sensor chip 31 to have simultaneous detection of various kinds of substances.

When the detection areas are disposed in the thickness direction of porous body 551, for example, a confocal microscope detects an object substance at each detection area.

A porous material for probe-immobilizing part 33 (133, 233) increases a specific surface area for immobilizing probes 5, i.e., increases an immobilizing amount of probes 5. Increase in immobilized probe 5 increases substances captured by probes 5, enhancing sensitivity of sensor chip 31 (31A, 31B).

Besides, the thickness of porous body 51 (551) of 100 μm allows signals to be limited within the focal length of a generally used fluorescence scanner, enhancing detection efficiency of sensor chip 31A (31B).

### Exemplary Embodiment 3

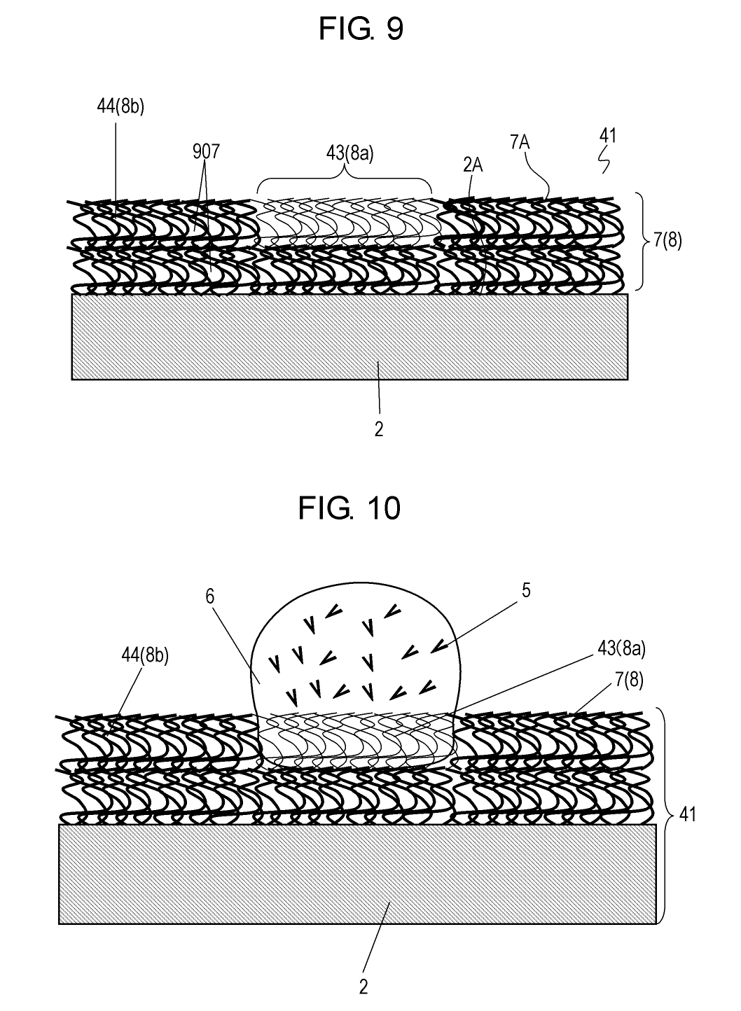

FIG. 9 is a cross-sectional view of sensor chip 41 in accordance with Exemplary Embodiment 3 of the present invention. In FIG. 9, components identical to those of sensor chip 1 shown in FIG. 1 according to Embodiment 1 are denoted by the same reference numerals. Sensor chip 41 includes fiber sheet 7 disposed on upper surface 2A of substrate 2. Fiber sheet 7 is a porous body having plural pores 907 therein. Fiber sheet 7 includes probe-immobilizing part 43 and liquid-spread-prevention part 44 which function similarly to probe-immobilizing part 3 and liquid-spread-prevention part 4 in accordance with Embodiment 1, respectively. Fiber sheet 7 is made of plural fibers 8 intertwined with each other. Fibers 8 have pores 907 between the fibers. Fibers 8 of fiber sheet 7 are made of amorphous silicon dioxide (hereinafter, simply referred to silicon dioxide). Fibers 8 intertwine with each other, i.e., are connected with each other to form a sheet that extends in parallel to upper surface 2A of substrate 2.

Fiber sheet 7 can be indirectly joined to upper surface 2A of substrate 2 via thermosetting resin adhesives and UV cure adhesives. Fiber sheet 7 can be directly joined with upper surface 2A by plasma activation.

In the case that substrate 2 is made of, for example, a material, such as silicon, quarts, or ceramics, containing silicon, heat applied to fibers 8 allows parts of the fibers to fuse thermally and allows fibers 8 to be easily bonded to upper surface 2A of substrate 2. The thermal fusion bonding does not require adhesive, and therefore, reduces cost. The “adhesive-free” thermal fusion bonding also reduces contamination on fibers 8 made of silicon dioxide, which is caused by highly volatile organic component included in adhesive.

Further, the following is another bonding. For example, a bonding layer of phosphorous silicate glass (PSG) film or a borophospho silicate glass (BSG) film is previously disposed on substrate 2 of silicon or quartz. The film is heated up to 1000° C. to cause fibers 8 made of silicon dioxide to be bonded to upper surface 2A of substrate 2 without thermal fusion. As described above, a film having a melting point lower than silicon dioxide and applied onto upper surface 2A of surface 2 prevents the structure from having conformational change due to thermal fusion of fibers 8. Fiber sheet 7 (fibers 8) is bonded to upper surface 2A of substrate 2 while maintaining a large surface area and a high pore ratio of fiber sheet 7.

For example, in the case that a bonding layer of polydimethylsiloxane (PDMS) is previously applied to upper surface 2A of substrate 2, the layer can be easily and firmly bonded to substrate 2 by pressing, with no need for thermal fusion of fibers 8 formed of silicon dioxide. This contributes to cost reduction.

Fiber sheet 7 can be formed such that each of fibers 8 of silicon dioxide is bonded to an adjacent fiber at least at one position. For example, when fiber sheet 7 is heated at a temperature higher than 1100° C., fibers 8 specifically neighboring fibers contacting each other are fused thermally. The sections of neighboring fibers contacting are bonded in a cooling process. Such formed fiber sheet 7 has fibers 8 bonded with each other. The structure where no separation occurs in silicon dioxide fibers 8 allows fiber sheet 7 to be easily handled.

Fiber sheet 7 may preferably have a thickness ranging from 10 to 100 μm. The fiber sheet having a thickness not less than 10 μm allows probe-immobilizing part 43 of fiber sheet 7 to have a large surface area per projecting area with respect to upper surface 2A of substrate 2. However, to make optical detection easy, the thickness of fiber sheet 7 should not be too large. Preferably, fiber sheet 7 has a thickness preferably not larger than 100 μm so that the large surface area can be effectively used for optical detection.

Fibers 8 having a thickness not smaller than 0.01 μm can easily increase the density of probes 5 immobilized onto probe-immobilizing part 43. For example, IgG antibodies are employed for probe 5. In consideration of the fact that an IgG antibody has a diameter of approximately 10 nm, the thickness of each of fibers 8 not less than 10 nm avoids steric effect of probe 5, increasing the number of probes 5 connectable to each of fibers 8.

As shown in FIG. 9, fibers 8 forming fiber sheet 7 include hydrophilic fibers 8a and hydrophobic fibers 8b. Hydrophobic fibers 8b are disposed on upper surface 2A of substrate 2, and hydrophilic fibers 8a are disposed on hydrophobic fibers 8b. That is, hydrophilic fibers 8a are disposed above upper surface 2A of substrate 2 via hydrophobic fibers 8b. Probe-immobilizing part 43 is made of hydrophilic fibers 8a. For example, fibers that preferably are used for hydrophilic fibers 8a are fibers having undergone a surface preparation by surface treatment agent for applying hydrophilicity to surfaces of silicon-oxide fibers 8. Hydrophilic fibers 8a are obtained by a method in which, for example, a carboxyl group or an epoxy group is applied to surfaces of fibers 8 with use of silane coupling agent. Similarly, hydrophobic fibers 8b are obtained by a method in which a methacryloxy group, an acryl group, or a fluoro group is applied to surfaces of fibers 8 with use of silane coupling agent.

FIG. 10 is a cross-sectional view of sensor chip 41 according to Embodiment 3 for illustrating a method of immobilizing probe 5 onto sensor chip 41. When probe solution 6 containing prove 5 is applied to probe-immobilizing part 43, liquid-spread-prevention part 44 of hydrophobic fibers 8b repels probe solution 6 and the spreading area of probe solution 6 is limited to probe-immobilizing part 43 made of hydrophilic fibers 8a. That is, probes 5 are immobilized not to liquid-spread-prevention part 44 but only to probe-immobilizing part 43. In this way, sensor chip 41 determines the area to which probes 5 are immobilized, controlling spatial configuration of probe-immobilizing part 43 accurately.

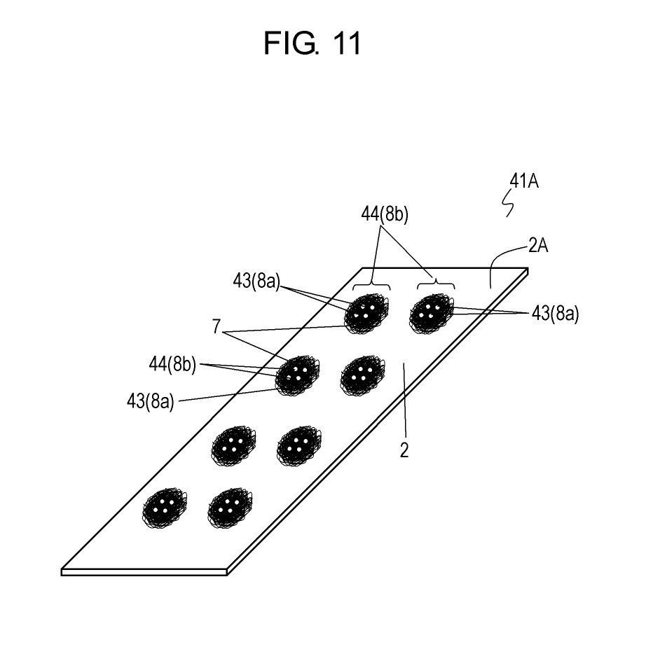

FIG. 11 is a perspective top view of another sensor chip 41A according to Embodiment 3. Sensor chip 41A includes plural fiber sheets 7 disposed on upper surface 2A of substrate 2. Each of fiber sheets 7, as shown in FIG. 11, has a circular shape and is disposed in parallel to upper surface 2A of substrate 2. Each of fiber sheets 7 includes plural probe-immobilizing parts 43. The structure allows spatial-capacity-controlled probe-immobilizing parts 43 to be disposed in a direction parallel to upper surface 2A of substrate 2 while maintaining a distance from substrate 2.

In sensor chips 41 (41A) according to Embodiment 3, reaction of probes 5 with an object substance effectively contacting probes 5 increases detection sensitivity of sensor chip 41 (41A). Besides, concentrating an object substance captured by probes 5 at high density allows a detector connected to sensor chip 41 (41A) to have enhanced efficiency in detecting a target substance, increasing detection sensitivity.

In the vicinity of the surface of substrate 2, liquid flow characteristics of a sample can decrease. However, in the structure where probe-immobilizing part 43 is away from substrate 2 by a distance, probe 5 and an object substance have contact reaction with no influence of the aforementioned decrease in liquid flow characteristics. This enhances reaction efficiency, increasing sensitivity of sensor chip 41 (41A).

Besides, the object substance can be concentrated by capturing an object substance by probe 5 with no dispersion in the depth direction of fiber sheet 7 (i.e., the direction perpendicular to upper surface 2A of substrate 2). This enhances sensitivity of sensor chip 41 (41A).

In sensor chip 41A, plural probe-immobilizing parts 43 are disposed on the same plane (i.e., on upper surface 2A of substrate 2). Probes 5 of different type immobilized to each of immobilizing parts 43 allows substances of various kinds to be detected simultaneously.

A method of using sensor chip 41 (41A) according to Embodiment 3 will be described below.

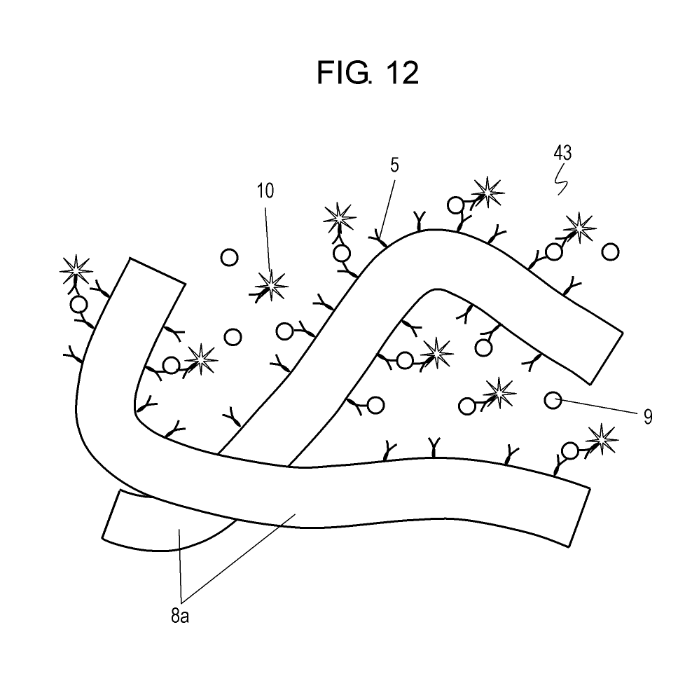

FIG. 12 is an enlarged view of sensor chip 41 (41A) for illustrating sample reaction observed in sensor chip 41 (41A), showing probe-immobilizing part 43 with probe 5 immobilized to the surface of hydrophilic fibers 8a. When object substance 9 is bonded to probe 5 and object substance 9 is captured by label 10, hydrophilic fibers 8a are labeled by label 10 as an indication of presence of object substance 9.

In the structure above, through the reaction of the solution containing target substance 9 and label 10 in pores formed between hydrophilic fibers 8a, label 10 is captured by hydrophilic fibers 8a via target substance 9. In probe-immobilizing part 43 after uncaptured label 10 is washed away, captured label 10 corresponding to the quantity of object substance 9 are left on hydrophilic fibers 8a. That is, the quantity of object substance 9 is detected by determining the quantity of captured label 10 on hydrophilic fibers 8a. As label 10, for example, fluorescent molecules, such as Cy3 and Cy5, may preferably be used. Irradiation of light corresponding to excitation wavelength for exciting each fluorescent molecule detects fluorescence emitted by the fluorescent molecule, determining the quantity of label 10.

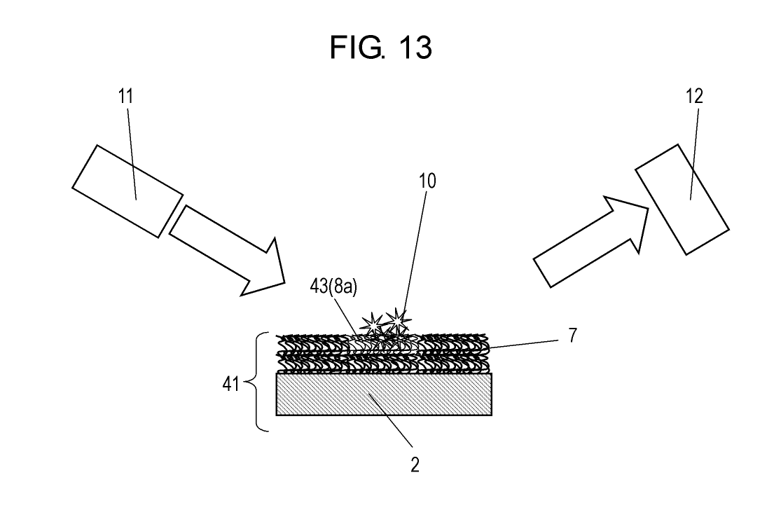

FIG. 13 is a cross-sectional view sensor chip 41 (41A) for illustrating detecting reaction of sensor chip 41 (41A). Although FIG. 13 illustrates the aforementioned fluoresce detection as an example illustrating detecting reaction of sensor chip 41 (41A), it is not limited to the method above.

For example, as described in the method above, fluorescent molecules as label 10 are captured by hydrophilic fibers 8a constituting prove immobilizing part 43 of fiber sheet 7.

Next, excitation light source 11 having a specific excitation wavelength irradiates captured label 10 allows the fluorescent molecules to emit fluorescence. The fluorescence emitted by fluorescent molecules is detected by fluorescence detector 12. For example, Cy3 is employed for the fluorescent molecule. In this case, excitation light irradiation is performed with laser having a wavelength of 532 nm supplied from excitation light source 11, and the fluorescence with a wavelength of 550 nm emitted by the molecule is detected by fluorescence detector 12. As for fluorescence detector 12, for example, a CCD and a photo multiplier tube are employed. A fluorescence filter that only allows certain wavelengths to pass through allows the fluorescence detection to enhance sensitivity.

As fluorescent molecules, for example, Cy3 and Cy5 may be employed. In the method above, plural fluorescent molecules having different fluorescence wavelengths may be captured by hydrophilic fibers 8a.

With the structure above, the quantity of the fluorescent molecules as label 10 captured by fiber sheet 7 of sensor chip 41 is determined based on an output of fluorescence detector 12. The quantity of target substance 9 is thus determined.

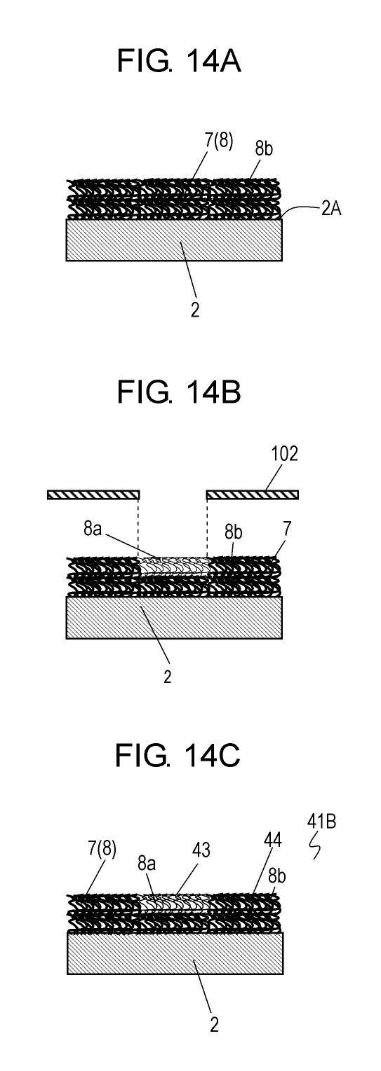

A method of producing sensor chip 41 according to Embodiment 3 will be described below. FIGS. 14A to 14C are cross-sectional views of sensor chip 41 according to Embodiment 3 for illustrating a method of producing sensor chip 41.

As shown in FIG. 14A, substrate 2 having fiber sheet 7 bonded to upper surface 2A thereof is prepared. The entire surface of fiber 8 of fiber sheet 7 is provided with surface treatment for applying hydrophobicity allows fiber sheet 7 to provide hydrophobic fibers 8b.

Next, as shown in FIG. 14B, light is irradiated to hydrophobic fibers 8b partly covered with mask 102 from a light irradiation device, such as a UV excimer laser. As mask 102, for example, a chromium shielding glass-plate is employed. Changing the shape for shielding allows a LTV irradiation area that undergoes UV irradiation to be changed, and also allows plural LTV irradiation areas to have light irradiation simultaneously.

When hydrophobic fibers 8b are irradiated with ultra violet light from a LTV irradiation device, the hydrophobic molecules applied to hydrophobic fibers 8b are removed due to oxidization. Having no longer hydrophobicity, fibers 8b change into hydrophilic fibers 8a. In this process, the depth of an irradiation part of fiber sheet 7 from which hydrophobicity is eliminated is determined by controlling energy of light irradiation.

Through the process above, as shown in FIG. 14C, a part with a predetermined depth from which hydrophobicity eliminated is produced. Probe-immobilizing part 43 is thus spatially determined. The aforementioned depth is not limited to a specific value.

Application of surface treatment with use of a silane-coupling agent; for example, application of a hydrophilic chemical functional group, such as an epoxy group and a carboxyl group, that forms covalent bonding with probe 5 allows the hydrophobicity-eliminated part to become probe-immobilizing part 43 of hydrophilic fibers 8a.

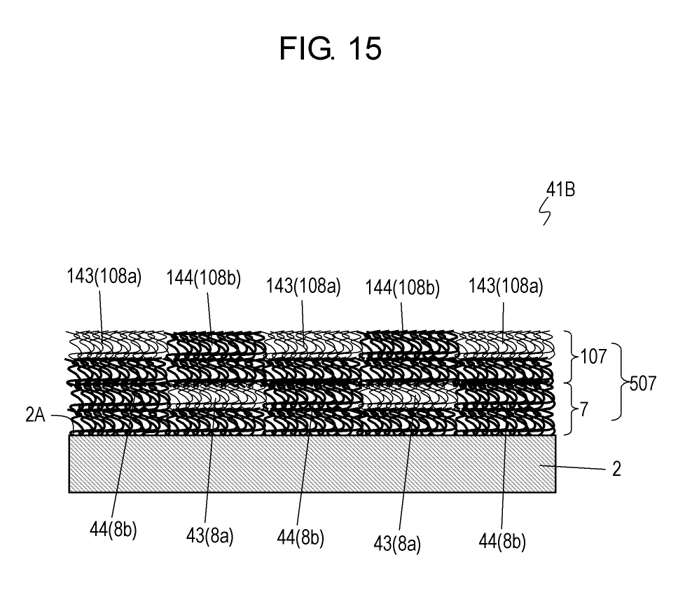

FIG. 15 is a cross-sectional view of still another sensor chip 41B according to Embodiment 3. In FIG. 15, components identical to those of sensor chip 41 shown in FIG. 9. Sensor chip 41B includes fiber sheet 507 disposed on upper surface 2A of substrate 2 instead of fiber sheet 7 of sensor chip 41 shown FIG. 9. Fiber sheet 507 is made of a porous body having plural pores 907 therein. FIG. 15 shows that hydrophilic probe-immobilizing parts 43 and 143 and hydrophobic liquid-spread-prevention parts 44 and 144 are selectively arranged in the thickness direction of fiber sheet 507. This configuration allows probe-immobilizing parts 43 and 143 to be spatially disposed in the structure. Probe-immobilizing part 43 is made of plural hydrophilic fibers 8a bonded with each other; similarly, probe-immobilizing part 143 is made of plural hydrophilic fibers 108a bonded with each other. In contrast, liquid-spread-prevention part 44 is made of plural hydrophobic fibers 8b; similarly, liquid-spread-prevention part 144 is made of plural hydrophobic fibers 108b. Fiber sheet 507 may be implemented by fiber sheet 7 disposed on upper surface 2A of substrate 2, and fiber sheet 107 disposed on upper surface 7A of fiber sheet 7. Fiber sheet 107 is made of a porous body having plural pores 907 therein. Fiber sheet 107 having probe-immobilizing part 143 and liquid-spread-prevention part 144 can be produced by a method similar to fiber sheet 7 including probe-immobilizing part 43 and liquid-spread-prevention part 44 shown in FIGS. 14A to 14C. The layered structure of fiber sheets 7 and 107 allows probe-immobilizing parts 43 and 143 to be disposed spatially.

The spatial arrangement of probe-immobilizing parts 43 and 143, that is, probe-immobilizing parts 43 and 143 for capturing an object substance are arranged in the thickness direction of sensor chip 41B, thereby increasing the detection area without extending sensor chip 41B horizontally. Increasing the detection area of sensor chip 41B allows simultaneous detection of various types of substances.

When the detection areas are disposed in the thickness direction of fiber sheet 7, for example, a confocal microscope detects an object substance at each detection area.

As described above, sensor chip 41 shown in FIG. 15 includes substrate 2, probe-immobilizing part 43 disposed on upper surface 2A of substrate 2, liquid-spread-prevention part 44 disposed around probe-immobilizing part 43 on upper surface 2A of substrate 2, probe-immobilizing part 143 disposed on upper surface 44A of liquid-spread-prevention part 44, and liquid-spread-prevention part 144 disposed around probe-immobilizing part 143. Probe-immobilizing parts 43 and 143 are configured to immobilize probe 5 by allowing probe solution 6 to be spotted to the probe-immobilizing parts. Liquid-spread-prevention parts 44 and 144 are configured to prevent the spotted solution from spreading from probe-immobilizing parts 43 and 143, respectively. Probe-immobilizing part 143 is disposed on the upper surface of liquid-spread-prevention part 44, and liquid-spread-prevention part 144 is disposed on the upper surface of probe-immobilizing part 43 to prevent the solution from spreading outside probe-immobilizing part 143. Probe-immobilizing part 43 is made of a porous body having pores therein. The porous body is fiber sheet 7 made of fibers 8a and 8b. Similarly, probe-immobilizing part 143 is made of a porous body having pores therein. The porous body is fiber sheet 107 made of fibers 108a and 108b.

In Embodiments 1 to 3, terms, such as “upper surface” and “above”, indicating directions merely indicate relative directions depending upon only relative positional relations of components, such as the substrate and the probe-immobilizing part, of the sensor chip, and do not indicate absolute directions, such as a vertical direction.

### INDUSTRIAL APPLICABILITY

A sensor chip according to the present invention is useful for proteomics research and bioassay technique in disease diagnosis.

### REFERENCE MARKS IN THE DRAWINGS

1, 21, 31, 41, 41A, 41B sensor chip
2 substrate
3 probe-immobilizing part (first probe-immobilizing part, second probe-immobilizing part)
4 liquid-spread-prevention part (first liquid-spread-prevention part, second liquid-spread-prevention part)
5 probe
6 probe solution
7, 107, 507 fiber sheet (first fiber sheet, second fiber sheet)
8 a plurality of fibers
8a fibers (first fibers)
8b fibers (second fibers)
9 target substance
33 probe-immobilizing part (first probe-immobilizing part)
34 liquid-spread-prevention part (first liquid-spread-prevention part)
43 probe-immobilizing part (first probe-immobilizing part)
44 liquid-spread-prevention part (first liquid-spread-prevention part)
108a, 108b fibers
133 probe-immobilizing part (second probe-immobilizing part)
134 liquid-spread-prevention part (second liquid-spread-prevention part)
143 probe-immobilizing part (second probe-immobilizing part)
144 liquid-spread-prevention part (second liquid-spread-prevention part)
907 pore

1. A sensor chip configured to be used together with probe solution containing a probe for capturing an object substance, the sensor chip comprising:
a substrate;
a first probe-immobilizing part disposed on an upper surface of the substrate, the first probe-immobilizing part being configured to immobilize the probe by allowing the probe solution to be spotted to the first probe-immobilizing part; and
a first liquid-spread-prevention part disposed around the first probe-immobilizing part so as to prevent the spotted probe solution from spreading from the first probe-immobilizing part,
wherein the first probe-immobilizing part is made of a first porous material having pores provided therein.

2. The sensor chip according to claim 1, wherein the first porous body comprises a first fiber sheet made of first fibers forming the pores.

3. The sensor chip according to claim 2, wherein the first fibers are made of amorphous silicon dioxide.

4. The sensor chip according to claim 2, wherein the first fiber sheet has a circular shape.

5. The sensor chip according to claim 1, further comprising:
a second probe-immobilizing part disposed on an upper surface of the first liquid-spread-prevention part, the second probe-immobilizing part being configured to immobilize the probe by allowing the probe solution to be spotted to the second probe-immobilizing part; and
a second liquid-spread-prevention part disposed around the second probe-immobilizing part so to prevent the spotted probe solution from spreading from the second probe-immobilizing part,
wherein, the second probe-immobilizing part is made of a second porous body having pores provided therein.

6. The sensor chip according to claim 5,
wherein the first porous body comprises a first fiber sheet made of first fibers forming the pores, and
wherein the second porous body comprises a second fiber sheet made of second fibers forming the pores provided therein.

7. The sensor chip according to claim 6, wherein the first fiber sheet is directly or indirectly joined to the upper surface of the substrate.

8. The sensor chip according to claim 6, wherein the second fibers and the first fibers are made of amorphous silicon dioxide.

9. The sensor chip according to claim 5, wherein the second liquid-spread-prevention part is disposed on an upper surface of the first probe-immobilizing part.

10. The sensor chip according to claim 1, further comprising:
a second probe-immobilizing part disposed on the upper surface of the substrate, the second probe-immobilizing part being configured to immobilize the probe by allowing the probe solution to be spotted to the second probe-immobilizing part; and
a second liquid-spread-prevention part disposed around the second probe-immobilizing part on the upper surface of the substrate so as to prevent the spotted probe solution from spreading from the second probe-immobilizing part.

11. The sensor chip according to claim 10, further comprising:
a first fiber sheet having a circular shape and including the first probe-immobilizing part and the first liquid-spread-prevention part; and
a second fiber sheet having a circular shape and including the first probe-immobilizing part and the first liquid-spread-prevention part.

12. The sensor chip according to claim 1, wherein the first liquid-spread-prevention part has one or more surface treatments performed thereon.

13. The sensor chip according to claim 1, wherein the substrate is made of at least one of glass, silicon, quartz, ceramics, resin, and metal.

14. The sensor chip according to claim 1, wherein the first liquid-spread-prevention part has hydrophobicity higher than the first probe-immobilizing part.
